(19) United States
(12) Patent Application Publication
(10) Pub. No.: US 2011/0305618 A1
(43) Pub. Date: Dec. 15, 2011

(54) METHOD FOR SYNTHESIS OF A RADIONUCLIDE-LABELED COMPOUND USING AN EXCHANGE RESIN

(75) Inventors: Keith Graham, Berlin (DE); Axel Rother, Berlin (DE); Dominic Franck, Berlin (DE)

(73) Assignee: BAYER SCHERING PHARMA AKTIENGESELLSCHAFT, Berlin (DE)

(21) Appl. No.: 13/141,584

(22) PCT Filed: Dec. 12, 2009
(86) PCT No.: PCT/EP2009/008901

§ 371 Date: Aug. 8, 2011

(30) Foreign Application Priority Data
Dec. 22, 2008 (EP) 08075961.6

(51) Int. Cl. C01B 7/20; C07C 231/12; B01J 19/00; C07C 213/08; C07D 471/04; C07D 487/04; C07C 231/24; C07C 213/10

(52) U.S. Cl. 423/249; 564/216; 564/219; 564/437; 564/443; 546/121; 544/281; 422/255

(57) ABSTRACT

The invention pertains to a method for eluting a radionuclide-label or a radionuclide-labeled compound using a solid phase extraction resin, to a device for performing such a method, and to a computer program for controlling such a device.

The invention pertains to a method for eluting a radionuclide-label or a radionuclide-labeled compound using a solid phase extraction resin, to a device for performing such a method, and to a computer program for controlling such a device.

### BACKGROUND OF THE INVENTION

Methods for the automated synthesis of radionuclide-labeled compounds with a solid phase extraction resin such as an anion exchange resin or a reversed phased resin have long been known in the art. Such methods have been applied in particular for generating radio-labeled compounds which can be used as tracer molecules for various kinds of biological applications. Such applications include positron emission tomography (PET), micro-PET or single photon emission computed tomography (SPECT), which is a diagnostic technique in nuclear medicine which makes use of radioactive, positron emitting isotopes that are linked to molecules of biological relevance.

Amongst the radionuclide-labeled compounds know to be used with PET are ¹⁸F-radiolabeled molecules as radiotracers, which are administered to a patient and the gamma radia20 tion emitted by the decay of the radioactive isotope is detected by a detection system, the so called PET scanner.

PET scans provide three-dimensional images that display the biological distribution pattern of a respective radio tracer in a cell, tissue or organism and thus allow the examination of biological processes in vivo.

Many radioisotopes that are used to label compounds used as tracers have a relatively short half-life. In particular, radionuclides used in PET scanning are typically positron emitting isotopes with short half lives such as carbon-11 (with a half life of about 20 minutes), nitrogen 13 (with a half life of about 10 minutes), oxygen-15 (with a half life of about 2 minutes), fluorine-18 (with a half life of about 110 minutes), iodine-131 (with a half life of about 8 days) and iodine-124 (with a half life of about 4.2 days). Therefore, it is desirable to provide a synthesis method for generating radionuclide-labeled compounds that can be performed quickly and with high yield. This is especially true for radionuclide-labeled compounds that are to be used for medical purposes, as described above. For example, ¹⁸F-labeled tracers used for PET need to be synthesized and purified as rapidly as possible.

Accordingly, it is desirable to provide a synthesis method that can be performed in a short time in order to increase the non-corrected radiochemical yield of the final product, the radionuclide-labeled compound.

Furthermore, it is imported when using an automated synthesis method to generate a radionuclide-labeled compound that the synthesis results, for example measured by the non-corrected radiochemical yield of the final product, can be consistently repeated using the same method. The methods for an automated synthesis know in the state of the art, however, have proven to result in the generation of products with an inconsistent yield.

### DESCRIPTION OF THE INVENTION

Accordingly, the problem underlying the present invention was to provide a method for eluting radionuclide-label or radionuclide-labeled compound on a solid phase extraction resin. The present invention allows for consistent yields, improved radioactivity yields as well as a short synthesis time.

The method for eluting comprises the step of removing or eluting a compound that is bound to a solid phase extraction resin by performing a pulsed elution with an eluent wherein the compound is a radionuclide-label or a radionuclide-labeled compound.

Optionally, the method of the present invention is used during the synthesis of a radionuclide labeled compound in which on a solid phase extraction resin is used.

The problem is solved by the present method, the present device, and by the present computer program, all of which are described in more detail below.

Specifically, the method according to the present invention for the synthesis, in particular the automated synthesis of a radionuclide-labeled compound using a solid phase extraction (SPE) resin comprises the step of removing or eluting a compound that is bound to a solid phase extraction resin by performing a pulsed elution with an eluent.

The term “automated synthesis” refers to a chemical synthesis that is performed without human intervention. In other words, it refers to a process that is driven and controlled by at least one machine and that is completed without the need of manual interference.

The pulsed elution, which will be explained in more detail below, surprisingly allows for both a consistently high yield of the radionuclide-labeled compound and for a shorter synthesis time.

The method can be performed with different solid phase extraction resins, as will be described below. Since different solid phase extraction resins can be used in the present method, the pulsed elution can occur at different steps of the automated synthesis. Specifically, the pulsed elution can be performed before or after a labeling reaction, in which a precursor molecule is labeled with a radionuclide in a reaction container to form a radionuclide-labeled compound.

In a preferred embodiment, the compound bound to a solid phase extraction resin that is eluted in a pulsed fashion can be a radionuclide-label that can be used for reacting with a precursor molecule to form a radionuclide-labeled compound. Alternatively, the compound that is bound to a solid phase extraction resin can also be a radionuclide-labeled compound that was generated by the reaction of a precursor molecule with a radionuclide label e.g. in a reaction container.

It is preferred that the radionuclide-label that is generated to react with a precursor molecule in order to label the precursor is bound to a solid phase extraction resin in the form of an anion exchange resin.

Furthermore, it is also preferred that the radionuclide-labeled compound generated by a reaction of a precursor molecule with a radionuclide label, e.g. in a reaction container, is bound on a solid phase extraction resin in the form of a reversed phase resin.

Accordingly, it is preferred to use a solid phase extraction resin in the present method that is an anion exchange resin for purifying a radionuclide-label. The solid phase extraction resin can also be a reversed phase resin for purifying a radionuclide-labeled compound, in particular using high performance liquid tomography (HPLC).

The solid phase extraction resin, e.g. in the form of an anion exchange resin or a reversed phase resin, may comprise or may be made of a whole range of different materials. Preferred are materials that are selected from the group consisting of silica and its derivatives, such as octadecyl-silica (monofunctional C18, trifunctional tC18), C8, tC2, C4, Phenyl, HLB (Hydrophilic-Lipophilic Balance) Sep-Pak Dry (anhydrous sodium sulfate) and magnesium silicate (Florisil®); Accell™ Plus CM (carboxylic acid salt), Accell™ Plus QMA (quaternary methylammonium), Alumina A (acidic), Alumina B (basic), Alumina N (neutral), amino propyl (NH₂), cyano propyl (CN), diol, WCX (weak cation exchange), MCX (medium cation exchange), SCX (strong cation exchange), WAX (weak anion exchange), MAX (medium anion exchange), SAX (Strong anion exchange), HILIC (Hydrophilic Interaction Liquid Chromatography) and DNPH-silica (acidified dinitrophenylhydrazine reagent coated on a silica sorbent). All of these materials are known, also in respect of their use in solid phase extraction cartridges containing the resin.

The present method can be practiced with practically any radionuclide-label. It is preferred that the radionuclide-label is chosen from the group consisting of Fluorine-18 [¹⁸F], Bromo77 [⁷⁷Br], Bromo-76 [⁷⁶Br], Oxygen-15 [¹⁵O], Nitrogen-13 [¹³N], Carbon-11 [¹¹C], Iodine-123 [¹²³I], Iodine-124 [¹²⁴I], Iodine-125 [¹²⁵I], Iodine-131 [¹³¹I], and radioactive metals, such as Gallium-67 [⁶⁷Ga], Gallium-68 [⁶⁸Ga], Yttrium-86 [⁸⁶Y], Yttrium-90 [⁹⁰Y], Lutetium-177 [¹⁷⁷Lu], Technecium-99m [^{99m}Tc], Technecium-94m [^{94m}Tc], Rhenium-186 [¹⁸⁶Re], Rhenium188 [¹⁸⁸Re], and Indium-111 [¹¹¹In]. More preferably, the radionuclide-label is Fluorine-18 [¹⁸F].

In the method of the invention, the elution is preferably performed with a solvent or eluent as appropriate for the solid phase extraction resin that is used and the compound bound thereon. The volume of the eluent with respect to the mass of the solid phase extraction resin usually has a ratio of 1:1 to 1:15. More preferably, the ratio is 1:2 to 1:10, even more preferred 1:2.5 to 1:5. The typical volume is approximately 2.5 times the mass of the resin. For example, a 100 mg resin can be eluted with a 250 μl of eluent (ratio volume eluent to volume of the SPE resin=2.5).

The elution can be performed at a temperature of between 10° C. to 100° C. Preferably, it is performed at between 20° C. to 50° C. In a preferred embodiment, the elution is performed at ambient temperature. It is also or additionally possible to heat the eluent used for eluting the resin, preferably to a temperature between 20° C. to 100° C., preferably to 20° C. and 50° C.

The elution of the solid phase extraction resin depends on both the kind of resin that is used and also on the compound that is to be eluted from the resin. The eluent can comprise or can be chosen from the group consisting of water (of various pH values), aqueous buffer solutions, lower alcohols, such as methanol, ethanol, propanol, and isopropanol, organic solvents, such as acetone, acetonitril (MeCN), tetrahydrofurane (THF), dichloro methane (DCM), dimethyl formamide (DMF), dimethylsulfoxide (DMSO), toluene, hexane, ether, ethyl acetate or mixtures of the above. If the eluent is or comprises water, the water can be of various pH values using different acids (e.g. HCl, H₂SO₄, H₃PO₄) for lower pH values, or different metal containing bases (e.g. alkali metal salts of carbonates, hydrogen carbonates, oxalates, hydroxides) or organic bases (e.g. ammonium hydroxides or hydrogen carbonates, tetraalkylammonium hydroxides or hydrogen carbonates, tetraalkylphosphonium hydroxides or hydrogen carbonates) for higher pH values. The eluent could also comprise or contain ionic liquids and/or chelating moieties, e.g. 18-crown-6 or Kryptofix 2.2.2., or mixtures thereof.

The central aspect of the invention is the pulsed elution of a solid phase extraction resin. This pulsed elution can be understood to consist of a sequence of a first, a second and a third period. During a first period, an eluent is applied onto the resin for elution of the compound from the resin. This first period is followed by a second period, during which no eluent is applied onto the resin. Instead, the eluent is allowed to incubate with the resin to which the compound is bound to allow for efficient elution of the compound from the resin. The eluate (i.e. the eluent and the compound that was previously bound to the resin) is then removed from the resin by a short (positive or negative) pressure period (third period) which can be caused by a means for performing a pulsed elution of the solid phase extraction resin, such as a pump (pressure pump or vacuum pump) or a flow regulator that is connected with the solid phase extraction resin to be eluted, e.g. with at least one coupling line, preferably with at least one valve configured to allow a pulsed elution. The third period, during which the means for performing a pulsed elution of the solid phase extraction resin is directly connected to the resin, e.g. a valve is opened to have a pressure by applied from a pump to the resin via a connecting coupling line, can be 10 to 100 seconds, preferably 30 to 50 seconds long.

Preferably, at least one other sequence of a first period in which the eluent is flowing into the resin for elution, followed by a second period in which no eluent is flowing into the resin and a third period for eluting the resin is performed in the method of the invention.

The first elution period can range between 0.1 to 8 seconds, preferably between 0.5 and 2 seconds in length. The second period, independently from the first period, can also last between 0.1 to 8 seconds, preferably between 0.5 and 2 seconds. The pressure applied for eluting the resin generally depends on the kind of resin used, the kind of eluent, etc. For example, a positive pressure of 1.5 bar (100 kN/m²) can be used.

In a preferred embodiment, at least the first and second, optionally also the third period is repeated at least once. Further repetitions of the sequence comprising a first, second, and optionally third period can be performed in the method of the invention if needed to elute more of the compound from the resin. If only the first and the second period are repeated, then a third period is applied after the repetition of the first and the second period has been performed. The number of repetitions is preferably 1 to 10, more preferably 3 to 5.

For example, in the method of the invention, the elution can be performed with a 1 ml solution of an eluent, using an “on cycle as” the first period of 1 second, followed by an “off cycle” as the second period of 1 second. This is preferably repeated 3 to 4 times. After the sequence as described has been performed, a valve in a coupling line that is connected to the solid phase extraction resin is opened, e.g. for 50 s.

The inventors have surprisingly found that the pulsed elution that is performed with the eluent incubating on the solid phase extraction resin during the second period as described above and its release in short cycles of pressure and time results in a more homogenous elution of the compound, as the elution solution can re-equilibrate on the column and elute higher amounts of the compound from the resin.

The method of the invention can be used for obtaining a large range of radionuclide-label or radionuclide-labeled compounds from at least one precursor molecule that is to be labeled, as will be recognized by a person of skill in the art.

In particular, the present method can preferably be used to synthesize radiolabeled compounds of four different groups:
a) The first group of compounds is described in the international patent application WO 2006/066104, which is hereby incorporated by reference. A very preferred group of compounds is represented by formula III.

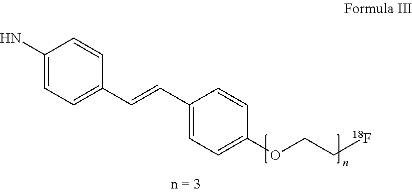

b) The second group of compounds are phenyloxyaniline derivatives as described in the international patent application WO 2008/028533 and U.S. Pat. No. 6,870,069, which are hereby incorporated by reference, and is represented by formula IV.

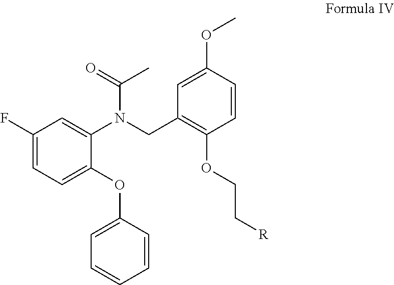

wherein R is a radionuclide as described above and herein and F is fluorine.

A particularly preferred compound of this group that can be obtained with the present method is N-[2-(2-[¹⁸F]-Fluoroethoxy)-5-methoxybenzyl]-N-(5-fluoro-2-phenoxyphenyl)-acetamide ([¹⁸F]-FEDAA), shown in formula II

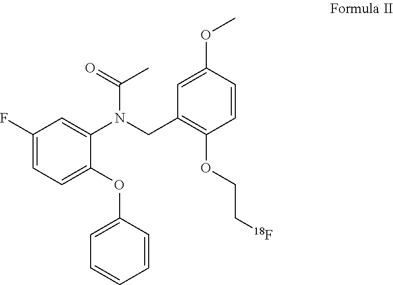

c) The third group of compounds is described in the international patent application WO 2008/022396, which is hereby incorporated by reference, and is represented by formula V.

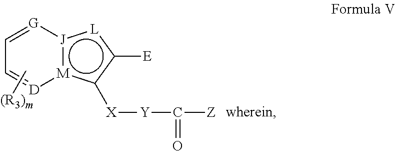

D, G, and L are independently selected from the group consisting of: CH, C and N, and J and M are independently selected from the group consisting of C and N provided that at least one of J and M is C, wherein at least two of D, G, M, J and L are N; X is selected from the group consisting of: O, NH, (CH₂)ₙ and S; Y is absent, or is selected from the group consisting of: O, NH and (CH₂)ₙ, and S;
Z is selected from the group consisting of: NR₁R₂ and aryl; R₁ and R₂ are independently selected from the group consisting of: hydrogen, C₁-C₁₀ alkyl, C₂-C₁₀ alkenyl, C₂-C₁₀ alkynyl, aryl and heteroaryl, each being optionally substituted with one or more of the following substituents: halogen, an C₁-C₆ alkyl; or R₁ and R₂, together with the nitrogen to which they are attached, form a heterocyclic ring having between 3 and 7 ring members, optionally substituted with one or more of the following substituents: halogen and C₁-C₆ alkyl; R₃ is selected from the group consisting of: halogen, C₁-C₁₀ alkyl and O—(C₁-C₁₀ alkyl), wherein the C₁-C₁₀ alkyl group is optionally substituted; E is an aryl group or a heteroaryl group, wherein each is substituted with one or more radionuclide label(s), e.g. F, or with one or more of the following substituents: C₁-C₆ alkyl, C₂-C₁₀ alkenyl, C₂-C₁₀ alkynyl, QC₁-C₁₀ alkyl, QC₂-C₁₀ alkenyl, QC2-C10 alkynyl, Q(CH2)p-Q-(CH2)qCH3 or Q(CH2)P-Q-(CH2)q-Q-(CH2)rCH3, each of which is substituted with one or more radionuclide label(s), e.g. ¹⁸F, and wherein p, q and r are, independently, integers between 1 and 3, and wherein Q is selected from the group consisting of: NH, O and S, m is a number between 0 and 3; n is a number between 1 and 4, wherein n in X is the same as or different to n in Y; with the proviso that R₃ is a fluoro substituent, or the group E comprises a fluoro substituent, or the group Z comprises a fluoro substituent, with the further proviso that E is not 4-fluorophenyl.

A particularly preferred compound of this group that can be labeled with the present method is PBR111, shown in formula Vb.

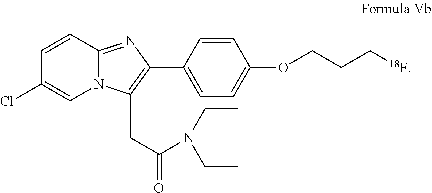

d) The fourth group of compounds is described in the international patent application WO 2007/134362, which is hereby incorporated by reference, and is represented by formula VI.

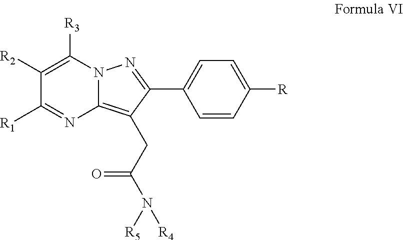

wherein:
R is alkyl substituted with a radionuclide, or alkoxy substituted with a radionuclide;
R₁, R₂ and R₃ are each independently H or a hydrophobic group; and R₄ and R₅ are each independently alkyl optionally substituted with halo, or alkoxy optionally substituted with halo.

A particularly preferred compound of this group that can be labeled with the present method is [¹⁸F]DPA-714: N,N-Diethyl-2-(2-[4-(2-fluoro-ethoxy)-phenyl]-5,7-dimethyl-pyrazolo[1,5-a]pyrimidin-3-yl)-acetamide, shown in formula VIb

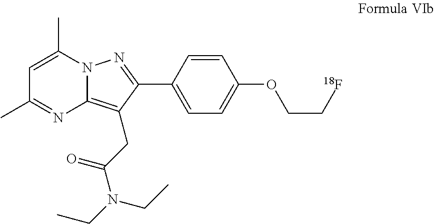

The synthesis of the radionuclide-labeled compounds shown above is preferably performed from a precursor that bears a leaving group instead of the radionuclide label. The radiolabeling reaction is preferably performed by substituting the leaving group with a radionuclide label.

It is particularly preferred that the radionuclide-labeled compound is an ¹⁸F-labeled compound, as they can be advantageously used in PET.

In the case that the radionuclide-labeled compound is a ¹⁸F-labeled compound, it is preferably selected from the group containing [18F]-fluorothymidine ([18F]-FLT), 6-[18F]-fluoro-LDOPA, [18F]-fluoromisonidazole, 1-(5-deoxy-5-[18F]-fluoro-α-D-arabinofuranosyl)-2-nitroimidazole ([18F]-FAZA), [18F]-fluoroethylspiperone, 16a-[18F]-fluoroestradiol, cis-4-[18F]-fluoro-L-proline, 2-[18F]-fluoro-1,3,5-tri-O-benzoyl-α-D-ribofuranose ([18F]-FMAU), [18F]-xeloda, 9-[(4[18F]-fluoro)-3-hydroxymethylbutyl]-guani-dine ([18F]-FHBG), 14-[18F]fluoro-6-thiaheptadecanoic acid ([18F]-FTHA), [18F]-fluoroethyl tyrosine ([18F]-FET), 2-[18F]fluoro-3-[2(S)-2-azetidinyl-methoxy]-pyridine tartrate ([18F]-FAP), [18]-fluoroacetate, [18F]fallypride, [18F]-flumazenil, [18F]-fluoro-altanserine, [18F]-fluoro-setoperone, N,N-diethyl[18F]-fluoro-methyltamoxifen, N-succinimidyl-4-[18F]-fluorobenzate ([18F]-SFB), and 2-(1,1dicyclopropen-2-yl)-6-([18F]-fluoroethyl)-methylamino)-naphthalene ([18F]-FDDNP). It is particularly preferred to use the method described herein to synthesis N-[2-(2-[18F]-Fluoroethoxy)-5-methoxybenzyl]-N-(5-fluoro-2-phenoxyphenyl)-acetamide ([18F]-FEDAA).

[18F]-FEDAA is particularly suited for the use in PET imaging to detect neuro information in a patient.

In a preferred embodiment, the present method can be performed such that the radionuclide-labeled compound is synthesized in a one-pot synthesis. A one-pot reaction is a chemical reaction that can be carried out in a single vial, to which all necessary reagents are subsequently added and no transfer of the reaction solution or parts of it into another vial is needed for subsequent chemical reactions to obtain the desired radionuclide-labeled compound. In this respect, for example, an [¹⁸F]-radiolabeling reaction with subsequent cleavage of protecting group or groups by addition of an acid or a base can be regarded as a one-pot reaction.

Furthermore, it is possible that the radionuclide-labeled compound is generated with the method of the invention which is a one-step synthesis method. A one-step reaction is a chemical reaction in which all necessary reagents can be mixed together at once and no subsequent addition of another reagent is required to obtain the desired radionuclide-labeled compound.

The problem underlying the present invention is also solved by device for eluting a radionuclide-label or radionuclide-labeled compound, in particular for performing a method as described above and herein. Such a device comprises or contains at least one cartridge with a solid phase extraction resin suitable for binding a compound, and at least one elution means or means for performing a pulsed elution of a compound from the solid phase extraction resin with an eluent. The elution means may be a pump or a flow regulator.

The pulsed elution of the compound from the solid phase extraction resin can either be done by
a) applying a pressure to the solid phase extraction resin using at least one gas, preferably an inert gas, such as helium, argon, nitrogen, or any mixture thereof, or by
b) applying a vacuum to the solid phase extraction resin.
   The pulsed elution of the solid phase extraction resin is performed by opening and closing at least one coupling line connecting the elution means with the solid phase extraction resin through at least one valve, such that either a pressure or a vacuum can be applied to the resin in a pulsed fashion, resulting in the pulsed elution of the compound that was bound to the resin. Accordingly, the elution means can be e.g. a vacuum pump, a (positive) pressure pump, or a flow regulator. Modules that contain a solid phase extraction resin that can be eluted by pressure or vacuum are known in the art and are commercially available.

The device may also comprise a reaction container for reacting a precursor with a radionuclide. Additional features of the device may be deduced in particular from the description of FIG. 3, which describes a preferred embodiment of the device according to the present invention.

The problem underlying the present invention is also solved by a computer program or a computer program product, in particular when stored on a storage device such a floppy disc, a USB stick or a CD, in particular when used on a computer, for controlling a device as described above and herein. Such a program is configured to allow for a pulsed elution of a cartridge containing a solid phase extraction resin for binding a compound, in particular for binding a compound as described above and herein, by controlling at least one pump for performing a pulsed elution of the solid phase extraction resin with an eluent.

In particular, the program controls the opening and closing of at least one valve for opening and closing at least one coupling line connecting an elution means such as a pump with a solid phase extraction resin from which a compound is to be eluted by applying a (positive) pressure or a vacuum (negative pressure) in a pulsed fashion with an eluent.

Further features of the device and the computer program are apparent from the description of the method given herein.

### FIGURES

FIG. 1 shows a reaction scheme for the radiosynthesis of [¹⁸F]-FEDAA.

FIG. 2 shows a flow diagram of steps of the synthesis of a typical ¹⁸F-labeled compound.

FIG. 3 shows a scheme of a device for an automated synthesis of a radionuclide-labeled compound with at least one solid phase extraction resin, which is especially suited for the automated synthesis of [¹⁸F]-FEDAA as a radionuclide-labeled compound.

FIG. 4 shows a table of the residual activity left on a QMA cartridge used within an automated synthesizer after using a non-pulsed elution method in the radio synthesis of [¹⁸F]-FEDAA.

FIG. 5 shows a table of the residual activity left on a QMA cartridge within an automated synthesizer after using a pulsed elution method in the radio synthesis of [¹⁸F]-FEDAA.

FIG. 6 shows a table of the residual activity left on a Chromafix C18 cartridge within an automated synthesizer after using a non-pulsed elution method in the radio synthesis of [¹⁸F]-FEDAA.

FIG. 7 shows a table of the residual activity left on a Chromafix C18 cartridge within an automated synthesizer after using a pulsed elution method in the radio synthesis of [¹⁸F]-FEDAA.

FIG. 8 shows UV and radioactivity (gamma) chromatograms for [¹⁸F]-FEDAA. HPLC of [¹⁸F]-FEDAA as a radionuclide-labeled compounds using a solid phase extraction resin in the form of an ACE C18 3μ 4.6×50 mm column; with a flow of 1 ml/min. 45% MeCN in water for 10 minutes and 95% MeCN in water for 10 minutes.

### DESCRIPTION OF THE FIGURES

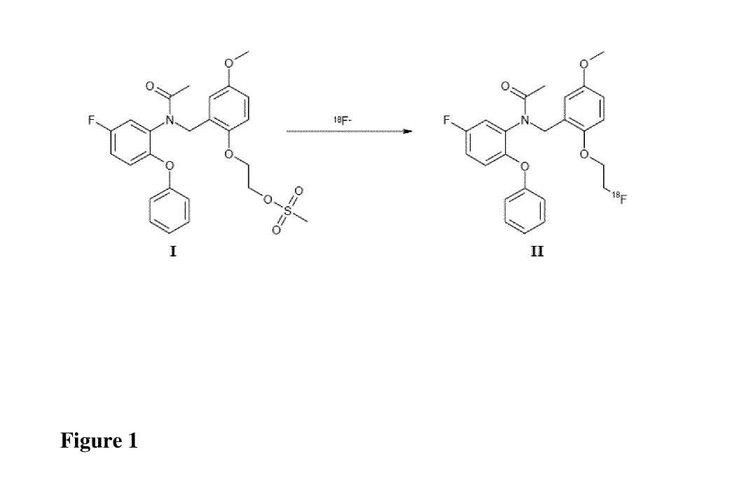

FIG. 1 shows a reaction scheme for the radio synthesis of [¹⁸F]-FEDAA (II) from compound (I) as a precursor molecule through labeling with a radionuclide label in the form of [¹⁸F].

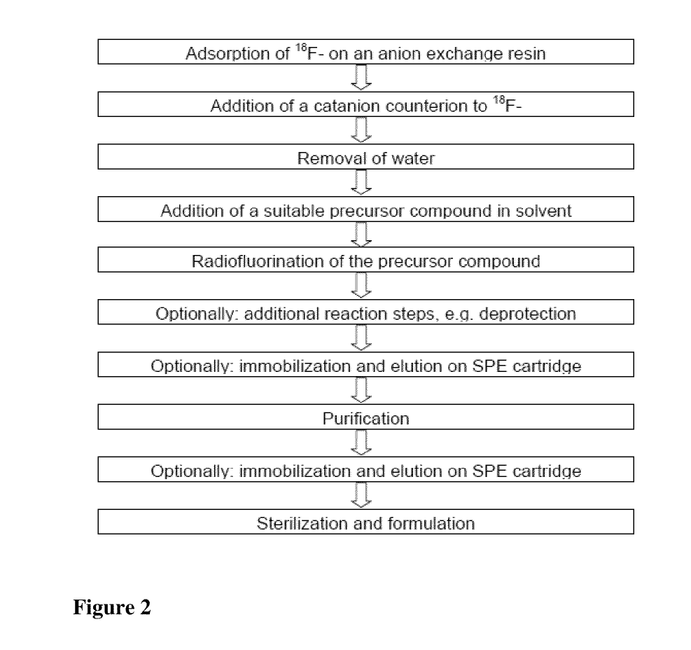

FIG. 2 shows a flow diagram of steps of a preferred synthesis method for generating a ¹⁸F-radiolabeled compound.

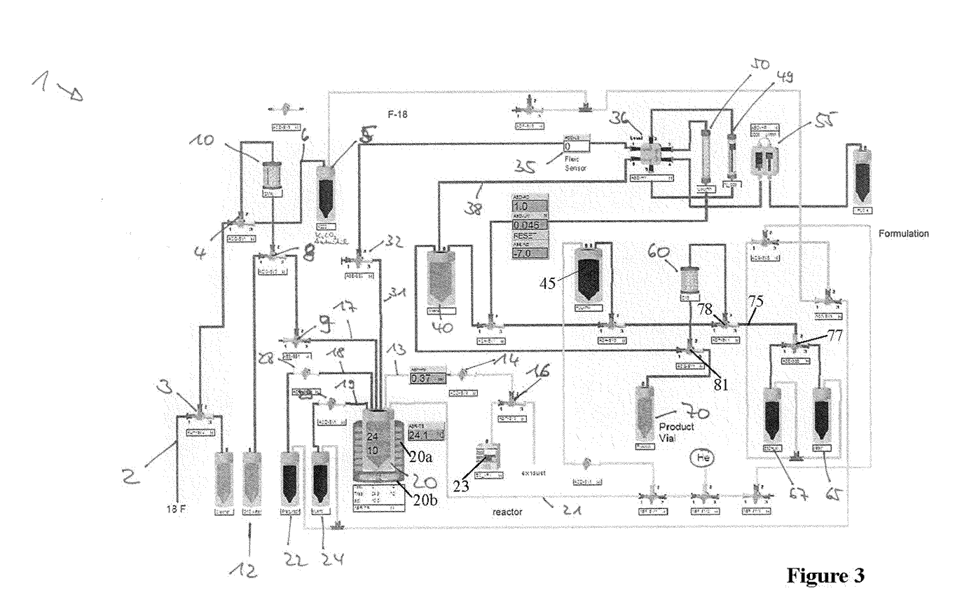

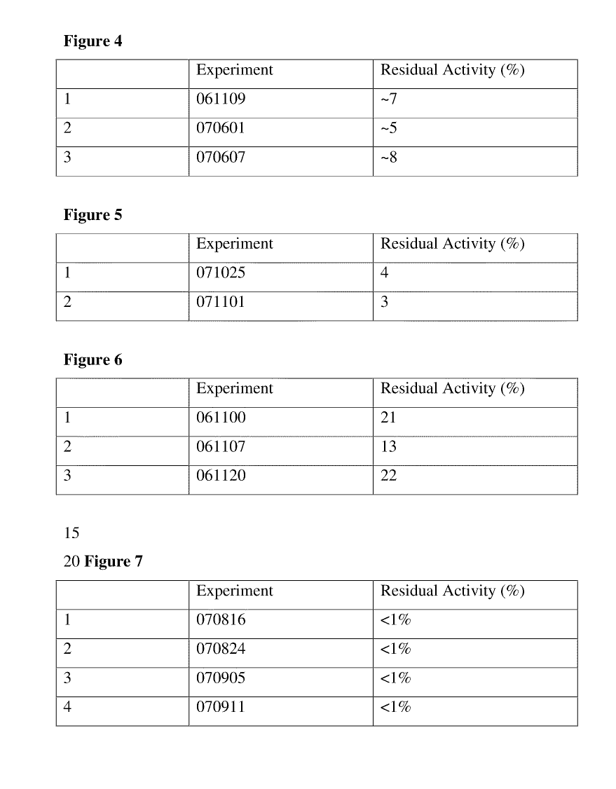

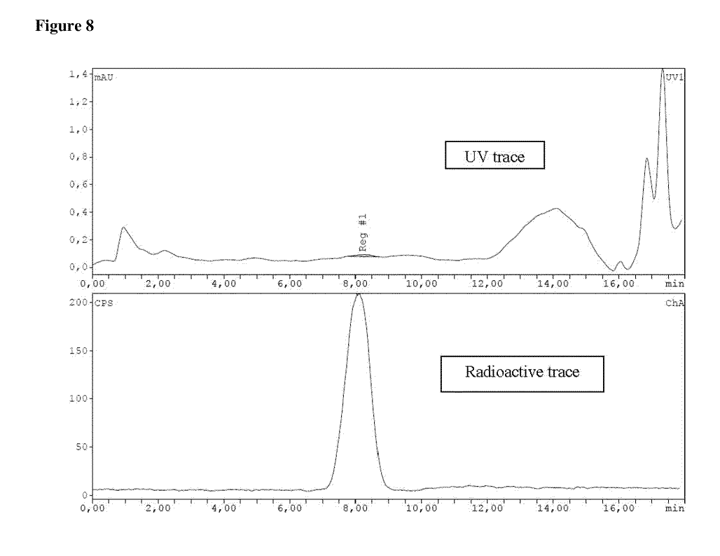

FIG. 3 shows a scheme of a device 1 for the automated synthesis of a radionuclide-labeled compound (a synthesis machine). In particular, the device shown can be used to perform a method for an automated synthesis of a radionuclide-labeled compound comprising at least one solid phase extraction (SPE) resin as described above and herein. The device is especially suited for the automated radiosynthesis of [¹⁸F]-FEDAA.

The use of the device 1 as shown in FIG. 3 will be described in conjunction with the synthesis of [¹⁸F]-FEDAA (II) from a precursor molecule (I) according to the reaction shown in FIG. 1. The precursor molecule (I) contains a mesylate group as a protective (leaving) group that is substituted in the reaction by an ¹⁸F-radionuclide label.

Details regarding chemicals and reaction parameters used, as far as they are not mentioned herein, can be obtained e.g. from Mäding et al., Annual report 2002, Institute of Bioinorganic and Radiopharmaceutical Chemistry, FZR-363, 40.

As a radionuclide-label, [¹⁸F]-fluoride ions contained in a target fluid are introduced onto a first solid phase extraction resin 10 in the form of a quaternary methylammonium resin (QMA) via a first feed line 2 that contains a first valve 3 and a second valve 4. The QMA column 10 allows for the extraction of [¹⁸F]-fluoride ions from the target fluid based on adsorption. The first solid phase extraction resin 10 might be positioned in a measuring chamber (not shown) for measuring the radioactivity on the first solid extraction resin. Here, 5 GBq [¹⁸F] were trapped on the first solid phase extraction resin 10 in the form of a QMA-cartridge that was preconditioned with 0.5 mol/l K₂CO₃-solution and washed with water.

The first solid phase extraction resin 10 is connected via a first coupling line 6 that also contains the second valve 4 with a first storage container 5. This first storage container 5 contains a solution (eluent) of Kryptofix 2.2.2 and potassium carbonate in aqueous acetonitrile. The content of the first storage container 5 can be applied onto the first solid phase extraction resin 10 using a vacuum or a carrier gas, such as nitrogen. Further, the first solid phase resin 10 is also connected to a reaction container 20 in which the labeling of a precursor molecule with a radionuclide-label (here, [¹⁸F]-fluoride) occurs. The first solid phase extraction 10 is con nected with the reaction container 20 via a second coupling line 17 that contains a third valve 8, and a fourth valve 9.

Form the first solid phase extraction resin 10, separated [¹⁸O]H₂O is removed from the first solid phase extraction resin 10 into the second storage container for [¹⁸O]H₂O 12 via a coupling line with the third valve 8.

As can be seen in FIG. 3, the reaction container 20 is connected via a third coupling line 18 with a seventh valve 28 to a third storage container 22 for the precursor molecule that is to be labeled with a radionuclide. The reaction container 20 is also connected to a forth storage container 24 for the eluent via a forth coupling line 19 with an eighths valve 29. Via the second coupling line 17 and the third coupling line 18, both the radionuclide-label (here, [¹⁸F]fluoride) and a precursor molecule (compound I of FIG. 1) can be brought into the reaction container 20 were the labeling of the precursor occurs, such that a radionuclide-labeled compound (here, [¹⁸F]-FEDAA) is formed. The educts are brought into the reaction container 20 using a vacuum or a gas, such as hydrogen, through the second 17 and third coupling lines 18. The reaction container 20 can be filled with an inert gas, such as helium, through a fifth coupling line 21. In order to release gas from the reaction container 20, a sixth coupling line 13 is connected to the reaction container 20 with a fifth valve 14 and a sixth valve 16, that allow to exhaust the reaction container 20.

The radionuclide-label (here, [¹⁸F]-fluoride) is eluted from the first solid phase extraction resin in the form of a QMA column 10 using a pulsed elution. As eluent, a solution of cryptofix K2.2.2./K₂CO₃ solution (1.0 mg K₂CO₃, 5.0 mg K2.2.2. dissolved in 0.2 ml H₂O and 0.8 ml acetonitrile (ACN)) is used, which is injected into the QMA column 10 for a first period of five seconds, followed by a second period (incubation period) of five seconds. Then, another injection of eluant into the QMA column 10 is performed for five seconds (another first period) followed by a five second incubation period (another second period). The first and second period are part of a pulsed elution sequence.

Specifically, the [¹⁸F] eluted from the QMA cartridge 10 was transferred into the reaction container 20. The elution was carried out in a pulsed pattern, by repeated closing and re-opening of the afferent tubing by the second valve 4 (ACG-SV1) with a cycle time of five seconds, as described above. The eluant is moved into the reaction container 20 via the second coupling line 17, where it is dried using a vacuum and nitrogen. The transfer of the eluant into the reaction container 20 is performed during a third period using a vacuum that is being generated by an elution means in the form of a vacuum pump 23. The vacuum pump 23 is connected to the reaction container 20 via the sixth coupling line 13 with the fifth valve 14 and the sixth valve 16, which need to be positioned together with the third valve 8 and the fourth valve 9 such that the vacuum generated by the vacuum pump 23 allows the elution of the radionuclide-label (here, [¹⁸F]-fluoride) from the first solid phase extraction resin 10 and its transfer into the reaction container 20.

Subsequently, a precursor (here, compound I shown in FIG. 1) is added to the reaction container 20 from the third storage container 22 via the third coupling line 18. The reaction mixture in the reaction container 20 is heated to a temperature of 120° C. and incubated for 5 minutes. Hereby, the radionuclide-labeled product, here [¹⁸F]-FEDAA, is formed. In order to obtain the required temperature of the reaction container 20, the device 1 contains a heating/cooling means 20a, and a stirring means 20b.

After the radionuclide-labeling of the precursor to form a radionuclide-labeled compound is completed in the reaction container 20, the product is moved form the reaction container 20 to a fluid sensor 35 via a seventh coupling line 31, which contains a ninth valve 32.

The fluid sensor 35 detects fluid in the seventh coupling line 31 and is arranged directly before a sample feet valve 36 for loading the synthesized radionuclide-labeled compound onto a second solid phase extraction resin in the form of a precolumn 49, from which it is being loaded onto a third solid phase extraction resin in the form of a reversed phase resin or a preparative HPLC-column 50.

The elution of the radionuclide-labeled compound, here [¹⁸F]-FEDAA, from the second 49 and/or third solid phase extraction resin 50 is in this example not performed in a pulsed manner. It is, however, also possible to configured the HPLC pump 55 such as to allow for a pulsed elution. Then, the pump 55 could be controlled by a program or computer program run on a computer to control the pump 55 and/or at least one valve to allow for a pulsed elution of the second 49 and third 50 solid phase extraction resin.

The radionuclide-labeled compound eluted from the HPLC-column 50 is then transferred into a vial 45 containing water (15 ml). The resulting solution is transferred onto a fourth solid phase extraction resin 60 in the form of a C18-column, where it is “trapped”. The C18 column is subsequently washed with water (2 ml). The elution of this C18-column 60 is also performed using a pulsed elution, through which the radionuclide-labeled compound is transferred into a product vial 70. For the elution of the [¹⁸F]-FEDAA from the C18-column 60, ethanol (1000 μl) stored in a sixth storage container 67 is used. The ethanol is brought onto the C18-column 60 in a pulsed manner as described above (with a cycle time of 1 second) through an eighth coupling line 75 with a tenth valve 77 and a eleventh valve 78. The elution of the C18-column 60 is performed by opening and closing a twelfth valve 81 in a pulsed manner three more times, and leaving open afterwards for 50 s.

### Example

### Synthesis of [¹⁸F]-FEDAA as a Radionuclide-Labeled Compound

[¹⁸F]-FEDAA was synthesized using a device for automated radiolabeling (here, radiofluorination) shown in FIG. 3.

5 GBq [¹⁸F] were trapped on a first solid phase extraction resin in the form of a QMA-cartridge that was preconditioned with 0.5 mol/l K₂CO₃-solution and washed with water. Subsequently, the [¹⁸F] was eluted with K₂₂₂/K₂CO₃ solution (1.0 mg K₂CO₃, 5.0 mg K₂₂₂ dissolved in 0.2 ml H₂O and 0.8 ml acetonitrile (ACN)) into the reactor that was preheated to 60° C. The elution was carried out in a pulsed pattern, by repeated closing and re-opening of the afferent tubing by a valve (ACG-SV1) with a cycle time of 5 s.

The solvent was evaporated by heating to 110° C. for 10 min under a weak vacuum aided by a gentle stream of dry nitrogen. After drying of the [¹⁸F]KF/K2.2.2., 2 mg of the precursor dissolved in DMF (600 μl) were added and heated at a reaction temperature of 120° C. After 5 min, the heating was stopped and the reactor was cooled to room temperature for 2 min. The reaction mixture was diluted with 3 ml of the eluent used for preparative HPLC (MeCN/water in a ration of 60/40) and the solution was applied to a preparative HPLC-separation.

The fraction containing [¹⁸F]-FEDAA was cut out and diluted with water and then trapped on a second solid phase extraction resin in the form of a Chromafix C18 cartridge. The cartridge was washed with water and [¹⁸F]-FEDAA was eluted with 1000 μl of ethanol in a pulsed pattern, by repeated closing and re-opening of the afferent tubing by a valve (ACG-SV1) with a cycle time of 1 s followed by an open valve of 50 s.

The total synthesis time from the addition of [¹⁸F] to the QMA-cartridge until the elution of the C18-cartridge takes 50 min and provides consistently 50% to 60% decay corrected radio-chemical yield, is comparison to the 2% to 60% reported in the literature (J. Med. Chem., 2004, 47, 2228-2235).

1. A method for eluting radionuclide-label or radionuclide-labeled compound on a solid phase extraction resin by performing a pulsed elution.

2. A method for synthesizing a radionuclide-labeled compound using a solid phase extraction resin, comprising
eluting a compound bound to a solid phase extraction resin from the solid phase extraction resin by performing a pulsed elution.

3. The method according to claim 2 that is an automated synthesis method.

4. The method according to claim wherein the compound bound to a solid phase extraction resin is
an radionuclide-label for reacting with a precursor molecule to form a radionuclide-labeled compound, or
a radionuclide-labeled compound that is generated by reacting a precursor molecule with an radionuclide-label in a reaction container.

5. The method according to claim 1, wherein the solid phase extraction resin comprises or is made of a material selected from the group consisting of silica and its derivatives, such as octadecyl-silica (monofunctional C18, trifunctional tC18), C8, tC2, C4, Phenyl, HLB (Hyrdrophilic-Lipophilic Balance) Sep-Pak Dry (anhydrous sodium sulfate) and magnesium silicate (Florisil®); Accell™ Plus CM (carboxylic acid salt), Accell™ Plus QMA (quaternary methylammonium), Alumina A (acidic), Alumina B (basic), Alumina N (neutral), amino propyl (NH2), cyano propyl (CN), diol, WCX (weak cation exchange), MCX (medium cation exchange), SCX (strong cation exchange), WAX (weak anion exchange), MAX (medium anion exchange), SAX (Strong anion exchange), HILIC (Hydrophilic Interaction Liquid Chromatography), and DNPH-silica (acidified dinitrophenylhydrazine reagent coated on a silica sorbent).

6. The method according to claim 1, wherein the solid phase extraction resin is an anion exchange resin for binding a radionuclide-label.

7. The method according to claim 1, wherein the solid phase extraction resin is a reversed phase resin for purifying a radionuclide-labelled compound, in particular using HPLC.

8. The method according to claim 1, wherein the radionuclide label is chosen from the group consisting of Fluorine-18 [¹⁸F], Bromo-77 [⁷⁷Br], Bromo-76 [⁷⁶Br], Oxygen 15 [¹⁵], Nitrogen-13 [¹³N], Carbon-11 [¹¹C], Iodine-123 [¹²³I], Iodine-124 [¹²⁴I], Iodine-125 [¹²⁵I], Iodine-131 [¹³¹I], and radioactive metals, such as Gallium-67 [⁶⁷Ga], Gallium-68 [⁶⁸Ga], Yttrium-86 [⁸⁶Y], Yttrium-90 [⁹⁰Y], Lutetium-177 [¹⁷⁷Lu], Technecium-99m [^{99m}Tc], Technecium-94m [^{94m}Tc], Rhenium-186 [¹⁸⁶Re], Rhenium188 [¹⁸⁸Re], and Indium-111 [¹¹¹In].

9. The method according to claim 1, wherein the ratio of the volume of an eluent for eluting the compound from the solid phase extraction resin over the mass of the solid phase extraction resin is between about 1:1 to about 1:15.

10. The method according to claim 1, wherein the solid phase extraction resin is eluted with an eluent that comprises or is chosen from the group consisting of water, aqueous buffer solutions, lower alcohols, such as methanol, ethanol, propanol, and isopropanol, organic solvents, such as acetone, acetonitril (MeCN), tetrahydrofurane (THF), dichloro methane (DCM), dimethylformamide (DMF), dimethylsulfoxide (DMSO), toluene, hexane, ether, ethyl acetate or mixtures thereof.

11. The method according to claim 1, wherein the pulsed elution comprises
a first period in which an eluent is flowing into the solid phase extraction resin for elution, followed by
a second period in which no eluent is flowing into the solid phase extraction resin, followed by
a third period in which eluate is flowing out the solid phase extraction resin.

12. The method according to claim 11, wherein the first period is between 0.1 seconds to 5 seconds, preferably between 0.5 seconds and 2 seconds long, the second period is between 0.1 seconds to 5 seconds, preferably between 0.5 seconds and 2 seconds long, and/or the third period is between 10 seconds to 100 seconds, preferably between 30 seconds and 50 seconds long.

13. The method according to claim 11, wherein the sequence of the first period and the second period is repeated at least once, preferably up to 10 times, more preferably up to 5 times.

14. The method according to claim 1, wherein the radionuclide-labeled compound is selected from the group consisting of compounds of formulas III, IV, V, and VI.

15. A device for eluting a radionuclide-label or radionuclide-labeled compound, in particular for performing a method according to claim 1, comprising
a cartridge containing a solid phase extraction resin for binding a compound, and
a means for performing a pulsed elution of the solid phase extraction resin.

16. The device according to claim 15, wherein the means for performing a pulsed elution of the solid phase extraction resin is a pressure pump, a vacuum pump, or a flow regulator.

17. A computer program, in particular when used on a computer, for controlling a device according to claim 15,
wherein the computer program is configured such as to allow for a pulsed elution of a cartridge containing a solid phase extraction resin for binding a compound; by controlling a pump for performing a pulsed elution of the solid phase extraction resin.
